(19) United States
(12) Patent Application Publication
(10) Pub. No.: US 2005/0131458 A1
(43) Pub. Date: Jun. 16, 2005

(54) Biodegradable embolic agents

(76) Inventors: Christopher D. Batich, Gainesville, FL (US); Matthew J. Eadens, Gainesville, FL (US); Robert A. Mericle, Brentwood, TN (US); Matthew V. Burry, Gainesville, FL (US); Courtney S. Watkins, Gainesville, FL (US); Swadeshmukul Santra, Gainesville, FL (US); Patrick Leamy, Downingtown, PA (US)

Correspondence Address: MILES & STOCKBRIDGE PC, 1751 PINNACLE DRIVE, SUITE 500, MCLEAN, VA, 22102-3833, US

(21) Appl. No.: 10/913,511

(22) Filed: Aug. 9, 2004

Related U.S. Application Data
(60) Provisional application No. 60/492,974, filed on Aug. 7, 2003.

(51) Int. Cl. A61B 17/08

(52) U.S. Cl. 606/214

(57) ABSTRACT

A flowable, biodegradable endovascular embolic composition effective for embolizing a vascular defect consisting essentially of: (a) a biocompatible, biodegradable polymer or polymeric material forming composition; (b) a biocompatible embolic solvent for the polymer or polymer forming composition capable of diffusion into mammalian tissue; (c) biocompatible magnetic particles responsive to a magnetic field; wherein: the polymer or polymeric forming material and solvent are present in the composition in amounts and relative proportions such that (1) the composition is deliverable to a vascular defect site and (2) upon delivery to the site, solidifies into an embolic mass; and the magnetic particles are present in the composition in an amount sufficient to enable the composition being deliverable to the vascular site by a magnetic field. Also disclosed are methods and articles of manufacture embodying the above-described composition.

### BACKGROUND OF THE INVENTION

1. Field of the Invention

This invention relates generally to biodegradable embolic compositions useful for the treatment of vascular defects such as cerebral arteriovenous malformations and aneurysms. This application claims the priority of provisional application Ser. No. 60/492,974, filed Aug. 7, 2004, the entire contents and disclosure of which are incorporated herein by reference.

2. Description of the Prior Art

Spontaneous intracranial hemorrhage can result from arteriosclerotic blood vessels, aneurysms, arteriovenous malformations (AVM), gliomas, and other known and unknown causes. Hemorrhaging from aneurysms alone is estimated to occur in 10 to 15 million Americans and nearly 70% of patients with AVM show hemorrhage at some point in their life. The treatment of aneurysms and AVMs has historically been a challenge to the neurosurgeon and neurologist. Except for advances within the past few decades, treatment options have been limited to surgical methods. As with any surgical procedure, complications and trauma are typical repercussions of invasive procedures.

Aneurysms have been traditionally treated with externally placed clips, or internally by detachable vasoocclusive balloons or an embolus generating vasoocclusive device such as one or more vasoocclusive coils. The delivery of such vasoocclusive devices can be accomplished by a variety of means, including via a catheter in which the device is pushed through the catheter to deploy the device. The vasoocclusive devices can be produced in such a way that they will pass through the lumen of a catheter in a linear shape and take on a complex shape as originally formed after being deployed into the area of interest, such as an aneurysm. In current techniques, the vasoocclusive devices take the form of spiral wound wires that can take more complex three-dimensional shapes as they are inserted into the area to be treated. By using materials that are highly flexible, or even super-elastic and relatively small in diameter, the wires can be installed in a micro-catheter in a relatively linear configuration and assume a more complex shape as they are forced from the distal end of the catheter.

For aneurysms, silicone or latex balloons and platinum Guglielmi detachable coils (GDC) are frequently used today. Particularly with balloons, these materials have been known to migrate, leading sometimes to aneurysm rupture. Complete occlusion of the aneurysms may also be difficult if the coils are not packed well enough or if leakage occurs around the balloon. The remnant flow can increase the size of the aneurysm and possibly lead to rupture. Thrombosis is also a key to aneurysm treatment, and these agents are not likely to be the best materials for promoting this response.

Today, options include less invasive procedures utilizing endovascular approaches. By way of a microcatheter, many different occluding materials or objects, known as embolic agents, are delivered to the vascular disease sites. Surgical methods, such as aneurysm clipping, are still employed today and usually are the most effective form of treatment. However, endovascular procedures have made many advances and these approaches have been used as the sole treatment method or as an adjunct to surgical resection or radiosurgery.

Adhesives that have been endovascularly delivered to help heal aneurysms include cyanoacrylates, gelatin/resorcinol/formol, mussel adhesive protein and autologous fibrinogen adhesive. Fibrin gels have also been used as sealants and adhesives in surgery, and hydrogels have been used as sealants for bleeding organs, and to create tissue supports for the treatment of vascular disease by the formation of shaped articles to serve a mechanical function. Catheters have commonly been used to introduce such therapeutic agents locally at diseased occluded regions of the vasculature to promote vessel healing. Typically a polymeric paving and sealing material in the form of a monomer solution, prepolymer solution, or as a preformed or partially preformed polymeric product, is introduced into the lumen of the blood vessel and positioned at the point of a stenosis. The polymeric material typically can incorporate additional therapeutic agents such as drugs, drug producing cells, cell regeneration factors, and progenitor cells either of the same type as the vascular tissue of the aneurysm, or histologically different to accelerate the healing process. See U.S. Pat. Nos. 5,580,568; 5,894,022; 5,888,546; 5,830,178; 6,113,629; 5,695,480 and 5,702,361.

However, many problems exist with the current embolic agents and much work needs to be done to improve them. N-butyl-2-cyanoacrylate (NBCA), a type of glue, is commonly used for the occlusion of AVMs. This material, combined with the iodinated poppyseed oil Ethiodol®, is injected in liquid form and polymerizes on contact with blood. Use of this glue has its drawbacks, however. Microcatheters, employed to deliver the material have been glued to vessel walls, and polymerized glue sometimes escapes the AVM and travels downstream to occlude healthy neural or pulmonary vessels. Due to the potential risks of NBCA traveling downstream and other difficulties, not all of the targeted areas within the AVM are typically embolized, which is key to embolization treatment for AVMs.

Hydrogels have also been used to form expanding, swelling stents, and as space-fillers for the treatment of vascular aneurysms in a manner similar to other types of mechanical, embolus generating vasoocclusive devices. In one such procedure, an aneurysm is treated by inserting a stent formed of a hydrogel material into the vessel, and then hydrating and expanding the hydrogel material until the stent occludes the vascular wall, sealing it from the parent vessel. Biodegradable hydrogels have also been used as controlled-release carriers for biologically active materials such as hormones, enzymes, antibiotics, antineoplastic agents, and cell suspensions.

Currently, the endovascular treatment of cerebral arteriovenous malformations (AVM) and aneurysms has become a popular option or adjunct to surgery. Many problems do exist, though, with the current materials that are used for embolization treatment. As noted above, N-butyl-2-cyanoacrylate (NBCA) glue is frequently unable to completely occlude AVMs and the same is true with coils or balloons for aneurysms. The development of improved or new agents is thus needed.

Recently, it has been suggested (U.S. Pat. Nos. 6,296,604 and 6,364,823) to incorporate a magnetic material in a liquid embolic agent comprising a precipitating polymer and a glue for delivery to and positioning within a vascular defect by an applied magnetic field. The compositions and methods disclosed by these patents, however, suffer from the disadvantage that the embolic mass formed at the site of the vascular defect is permanent and non-biodegradable. Alksne, “Iron-acrylic Compound for Stereotactic Aneurysm Thrombosis.” J. Neurosurg. 47:137-141 (1977) discloses injecting an iron-acrylic mixture into the dome of an aneurysm, and holding the mixture in place with a magnet inside the body. Gaston et al., “External Magnetic Guidance of Endovascular Catheters with Superconducting Magnet: Preliminary Trials” J. Neuroradiol. 15: 137-147 (1988) discloses delivering magnetic particles with an external source magnet. Evans, U.S. Pat. No. 5,702,361 “Method of Embolizing blood Vessels” discloses various embolizing agents including polymers and/or adhesives. Granov et al., U.S. Pat. No. 5,236,410, “Tumor Treatment Method,” discloses the use of magnetic materials in tumor treatment.

It is an object of the invention to provide novel biodegradable embolic compositions that can be delivered to the site of a vascular defect by an applied magnetic field.

### SUMMARY OF THE INVENTION

The above and other objects are realized by the present invention, one embodiment of which relates to a flowable, biodegradable endovascular embolic composition effective for embolizing a vascular defect consisting essentially of:
(a) a biocompatible, biodegradable polymer or polymeric material forming composition;
(b) a biocompatible embolic solvent for the polymer or polymer forming composition capable of diffusion into mammalian tissue;
(c) biocompatible magnetic particles responsive to a magnetic field;
   wherein:
the polymer or polymeric forming material and solvent are present in the composition in amounts and relative proportions such that (1) the composition is deliverable to a vascular defect site and (2) upon delivery to the site, solidifies into an embolic mass; and
the magnetic particles are present in the composition in an amount sufficient to enable the composition being deliverable to the vascular site by an applied magnetic field.

A second embodiment of the invention concerns a method of embolizing a vascular defect comprising introducing the above-described composition into the vascular defect under the guidance of an applied magnetic field and positioning the composition therein with the applied magnetic field under conditions wherein and until the composition solidifies into an embolic mass.

An additional embodiment of the invention involves the incorporation of a physiologically compatible bioactive agent, such as a drug, for example, in the embolic composition.

Another embodiment of the invention relates to articles of manufacture comprising the above-described composition.

### DETAILED DESCRIPTION OF THE INVENTION

The present invention relates to a flowable, magnetic embolic agent which can be delivered, e.g., injected through a syringe or catheter in a blood vessel to the site of a vascular defect and positioned therein by an applied magnetic field. The flowable agent solidifies when in contact with tissue such as blood or muscle, usually, but not exclusively, by diffusion of the biocompatible solvent into surrounding tissue or dissolution into blood. The solidified mass forms a biodegradable matrix, which can also be used, if desired, for the delivery therein of a bioactive agent such as, e.g., a drug. Magnetic particles, which are also degradable, are included to deliver the agent to the desired vascular defect site by an applied magnetic field and to hold the agent in place until solidification occurs.

Suitable embolic agents for the practice of the invention include any suitable biodegradable, biocompatible polymer or polymeric material forming material that is capable of occluding a vascular defect when introduced endovascularly into the site of the defect such as, for example, cellulose acetate (CA), polylactic acid (PLA), poly(glycolic acid) (PGA), copolymers of the PLA and PGA, and polycaprolactone (PCL).

When used in embolotherapy CA is dissolved in a solvent, such as dimethyl sulfoxide (DMSO), e.g., and is injected in liquid form through a microcatheter. DMSO solvent may be injected in a small amount (0.05-0.10 mL) to irrigate the microcatheter prior to injection of the CA/DMSO solution. When the solution is injected in the bloodstream, DMSO is soluble whereas the CA is not and the polymer precipitates out of the solution as a soft, solid mass. Typical injection mixtures are composed of 250 mg of CA, between 800 and 2250 mg of bismuth trioxide, and between 3 and 7 mL of DMSO [Tokunaga, K., K. Kinugasa, S. Mandai, A. Handa, N. Hirotsune, and T. Ohmoto. “Partial thrombosis of canine carotid bifurcation aneurysms with cellulose acetate polymer.” Neurosurgery. 42: 1135-1144 (1998); Neurosurgery. 44(5): 981-990 (May 1999), and Yang et al., Surgical Neurology. 55: 116-122 (2001)]. The bismuth trioxide is used as a radiopaque material for fluoroscopy. When injected slowly enough, at 0.5 mL over 30 seconds, DMSO does not show appreciable angiographic or pathologic effects.

Polylactic acid (PLA), also referred to as polylactide, is one of the most popular materials used for biodegradable applications. The polymeric form is synthesized from lactide cyclic monomer. A unique property of PLA is the stereochemistry of the structure. Three basic forms are possible, poly (D-lactic acid), poly (L-lacticacid), and poly(DL-lactic acid), with many variations of the racemic, or mixed copolymers of the DL polymer. The DL form usually is atactic, showing no regular repeating structure, and thus can form amorphous polymers whereas the other two forms are isotactic and have semi-crystalline characteristics, typically around 35% crystallinity. Due to the crystallinity of poly(L-lactic acid), it has better mechanical properties than the atactic form. Also, the L-form degrades at a much slower rate, typically between 20 months to 5 years compared to 6 to 17 weeks for the DL-form, although the degradation depends on the local environment.

Other typical biodegradable materials similar to PLA include poly(glycolic acid) (PGA), copolymers of the PLA and PGA, and polycaprolactone (PCL). These synthetic biopolymers exhibit good mechanical properties. The degradation products, such as glycolic acid for PGA, are also non-toxic and easily metabolized. These polymer types have frequently been used as surgical screws or degradable sutures. Recently, they have been utilized for drug delivery. As the polymer slowly degrades within the body, a trapped chemotherapeutic agent can diffuse into the immediate tissue.

Degradation of PLA takes place by hydrolysis of the ester linkage. This process is acid-, base-, and enzymatically-catalyzed. The cleavage of the ester link leaves a remaining carboxylic acid end that can further catalyze hydrolysis elsewhere (autocatalysis). A suitable solvent for PLA, PGA, copolymers of the PLA and PGA, and PCL for endovascular injection is ethyl lactate, which is produced from sugar fermentation.

Suitable solvents for the practice of the invention include any solvent capable of dissolving the biocompatible polymer or polymeric material forming composition, which is miscible or soluble in aqueous compositions (e.g., blood) and is capable of diffusing into mammalian tissue, e.g., DMSO, ethanol, ethyl lactate, acetone, N-methylpyrrolidone, triethylcitrate, and other liquid esters of natural products.

Magnetic Embolic Agents.

Previous to this study, little published work is available that evaluates the use of a magnetic embolic agent for endovascular drug delivery or the treatment of cerebral AVMs or aneurysms. Alksne and Fingerhut [Bulletin of the Los Angeles Neurological Societies. 30: 153-155 (1965)] and Alksne and Smith, supra, developed the idea of the use of a magnetic agent to improve the thrombosis of aneurysms. The first work used carbonyl iron powder suspended in human serum albumin that was held in place by an external magnet attached to the skull. However, clotting took five days, some magnetic pieces fragmented off, and the patient needed to return to the operating room to remove the magnet. In a subsequent study, the material used was the same carbonyl iron powder but instead suspended in a liquid methyl methacrylate monomer that is polymerized from catalysis by methyl methacrylate-n-butyl methacrylate polymer (Alksne and Smith, 1977). The embolic becomes a non-fragmenting solid in 30-60 minutes.

Magnetite and Forces Due to Field Gradients

The use of magnetically-guided particles and devices are seen in many applications such as the aforementioned magnetic embolic agents, intravascular catheter guidance [Frei et al., Medical Research Engineering. 5(4): 11-18 (1966)] and targeted drug delivery [Gupta and Hung, International Journal of Pharmaceutics. 59: 57-67 (1990)]. Frequently, these types of microparticles are made from iron oxide (Fe₃O₄), also known as magnetite. Although the chemical formula for magnetite is Fe₃O₄, it is often written as FeO·Fe₂O₃ because it consists of a 2 to 1 molar ratio of Fe³⁺ to Fe²⁺. The easiest preparation method of magnetite is precipitation from ferric and ferrous salts such as FeCl_{3 and FeCl}₂ [Fahlvik et al., Investigative Radiology. 25(2): 113-120 (February, 1990 and Molday and MacKenzie, Journal of Immunological Methods. 52(3): 353-367 (August, 1982)]. These particles can be made relatively small with diameters around 10 to 15 nm.

In Vitro Models

Numerous in vitro models have been fabricated for the experimental testing of cerebral AVMs and aneurysms. In vitro models for testing of embolic materials are generally a parallel flow circuit with an AVM branch and a Starling resistor branch to mimic the normal brain vasculature [Bartynski et al., Radiology. 167:419-421 (1988); Kerber et al., American Journal of Neuroradiology. 18: 1229-1232 (August, 1997); Park et al., American Journal of Neuroradiology. 18: 1892-1896 (November, 1997). The actual AVM model itself is some form of dilated tubing or shaped silicone filled with mesh, foam, or springs to mimic the nidus of an actual human AVM.

The invention is illustrated by the following non-limiting examples.

### EXAMPLES

Three magnetic embolic agents, NBCA/Ethiodol®, cellulose acetate (CA)/dimethyl sulfoxide (DM50), and poly-DL-lactide (PLA)/ethyl lactate, were developed for the tests described below. Oleate-coated and non-coated magnetite (Fe₃O₄) were added to these mixtures. Only the PLA is generally considered a biodegradable material because of its rate of degradation. The viscosity for these solutions versus shear rate was determined and a settling test for dispersion characteristics was conducted. The magnetic agents were then injected via a microcatheter into an in vitro dynamic flow system to evaluate the efficacy of the system.

NBCA/Ethiodol® Solutions.

NBCA was obtained from 3M (St. Paul, MIN) as the product Vetbond™. The solvent employed was Ethiodol® (Savage Laboratories®, NY). The oleate-coated magnetite (Fe₃O₄-oleate) component of the solution was prepared from the following materials: ferric chloride hexahydrate (FeCl₃₆H₂O), ferrous chloride tetrahydrate (FeCl₂₄H₂O), sodium hydroxide (NaOH), sodium chloride (NaCl), sodium oleate, and hydrochloric acid (HCl). Non-coated magnetite, iron (II, III) oxide (Fe₃O4), and glacial acetic acid (GAA) were also employed.

Cellulose Acetate/DMSO Solutions.

Cellulose acetate [39.7% acetyl content, viscosity of 114 Poise by ball-drop method of ASTM D 1343 in powder form] and DMSO were employed. The magnetite components were as described above.

Polylactic Acid (PLA)/Ethyl Lactate Solutions.

Poly-DL-lactide (Purasorb®, molecular weight 115,000) and ethyl lactate solvent were employed. The magnetite components were also the same as discussed above.

In Vitro Data Acquisition Flow System.

The materials used in the in vitro flow system were as follows: Masterflex® variable speed peristaltic pump, 0.25″ inner diameter Tygon® tubing, quick disconnect fittings, 0.25″ inner diameter latex tubing, 1/16″ Tygon® tubing, Intramedic® 0.58 mm inside diameter polyethylene tubing, 23-gauge needles, 3-mL syringes, sheath introducer, and a reservoir.

The simulated blood fluid (SBF) used for flow, experiments was comprised of the following materials: poly(vinyl alcohol) (PVA) with a molecular weight of 93,400, sodium chloride (NaCl), boric acid, and sodium tetraborate decahydrate.

Data Acquisition.

The materials and equipment for the data acquisition component of the flow system are as follows: a Gateway® E3100 computer, a Multifunction I/O data acquisition board (Model PC-LPM-16/PnP) (National Instruments®), NI-DAQ software Version 6.7 (National Instruments®), Lab VIEW™ 5.1 software (National Instruments®), an Archer breadboard (Radio Shack®), 50-pin ribbon cable, silicon pressure sensors with a range of 0 to 7.3 psi (MPX5050 series, Motorola, and a flow sensor with a range of 60 mL/min to 1,000 mL/min (Model 101T, McMillan Company)).

AVM and Aneurysm Models

The AVM in vitro model was made from open-celled polyurethane foam with dimensions of 4.5 cm by 3 cm by 1 cm (Stephenson & Lawyer, Inc.), two glass plates with dimensions of 10 cm by 10 cm by 0.5 cm, silicone (DAP, Inc.), insulation from 14-gauge wire, 0.25″ inner diameter Tygon® tubing, and quick disconnect fittings. The aneurysm model was constructed using all previously stated materials for the AVM model minus the foam and wire insulation.

### Methods

Magnetite or maghemite particles do not disperse well in a non-polar solvent without a surfactant or other treatment to make the surface hydrophobic and compatible with the solvent. Oleic acid works very well as a preliminary surface treatment for the polar magnetic particles to allow them to disperse very well in the solvent/polymer system. This produces a homogeneous mixture of particles in the liquid and avoids significant clumping or aggregation which is otherwise observed. This smooth dispersion behaves well in a magnetic field since there is a consistent attraction to the fluid, and no areas of significantly enhanced attraction.

The surfactant employed to coat the magnetic material may be any biocompatible surfactant that functions to impart a hydrophobic surface to the normally hydrophilic surface of thereof. The hydrophobicity of the surface of the magnetic material enhances its compatibility with the solvent and polymer, thereby facilitating its dispersion in the liquid mixture and avoiding settling out and/or aggregation thereof. The surfactant is preferably an unsaturated fatty acid; most preferably an 18 carbon atom fatty acid, e.g., oleic acid, linoleic acid or linolenic acid. These acids may be used in a form of salt, preferably a metallic salt, and more preferably an alkaline metal salt, such as the sodium salt, and the ammonium salt. The fatty acid salt coated magnetic particle fluid is a stable suspension of magnetic particles with a particle size, normally less than 300 A, in a carrier fluid. The suspension does not settle out under the influence of gravity or even of a magnetic field. The magnetic fluid responds to an applied magnetic field as if the fluid itself had magnetic characteristics.

Preparation of Oleate-Coated Magnetite Particles

The synthesis of the magnetite particles was completed using a procedure adapted from Gruttner et al. [“Preparation and characterization of magnetic nanospheres for in vivo application.” Scientific and Clinical Applications of Magnetic Carriers. Eds. U. Hafeli, W. Schutt, T. Teller, and M. Zborowski. New York: Plenum Press, 1997].

First, 3.02 g of FeCl₃·6H₂O and 1.28 g of FeCl₂·4H₂O was dissolved in 30 mL of deionized water. The solution was then placed in a double-walled beaker with water bath temperature control set at 67° C. While the solution was stirred, two molar NaOH was added dropwise to precipitate iron oxide, Fe₃O₄, and the pH was monitored using a pH probe. The final pH was approximately 10.85. The mixture was then washed with 102 molar NaCl, centrifuged for 15 minutes, and the supernatant was drained off. The washing and centrifuging process was completed a total of four times in order to wash the precipitate of any remaining ions. The magnetite particles were then suspended in the NaCl solution by sonication. The particles were sonicated for 10 minutes at level 4, 50% duty cycle in an ice bath to keep the mixture from heating.

The concentration of particles suspended in NaCl solution was calculated in terms of grams Fe₃O₄ per milliliter of solution. A 1 mL sample of solution is evaporated in an aluminum dish. The concentration is equal to the dry weight of the sample per milliliter of solution, under the assumption that the weight of the NaCl salt is negligible. The total amount of magnetite in the solution is equal to the product of this concentration and the total volume of solution.

The particles were then coated with oleate using a procedure adapted from U.S. Pat. No. 4,094,804. Sodium oleate was added in a ratio of 0.0153 g of Na-oleate to 0.01833 g of Fe₃O₄. This ratio was selected from previous experimental determination due to the optimum dispersion characteristics thereof. This solution was placed in an incubator for 80 minutes at 40° C. Then 0.1 molar HCl was added dropwise until the pH equaled 5.58. The particles were centrifuged and the supernatant was discarded. To remove any remaining salts, the particles were washed with deionized water and centrifuged. This washing procedure was completed twice. After discarding the final supernatant, the olcate-coated magnetite was placed in a freeze-dryer overnight.

Preparation of Solutions.

The following formulations were used in the viscosity measurements and flow system experiments described below. Hereafter, the oleate-coated magnetite will be denoted by MAG-oleate and the non-coated magnetite will be denoted simply by MAG.

NBCA/Ethiodol® Solutions

NBCA was mixed with Ethiodol® in a 1:1 ratio (0.5 mL each). Upon initial observation with addition of oleate-coated magnetite, the solution polymerized within 1-2 minutes and thus the addition of glacial acetic acid (GAA) was necessary to slow the polymerization. A 30 μL portion of GAA (3% by volume) was added to the solution. Then either 50 mg of MAG-oleate or 50 mg of MAG was added and stirred with a glass rod to disperse the particles prior to use.

Cellulose Acetate/DMSO Solutions.

The solutions made were variations of those disclosed by Tokunaga et al. [Journal of Clinical Neuroscience. 7(S1): 1-5 (2000). In a small vial (approximately 10 mL) 125 mg of CA was dissolved in 4.5 mL of DMSO. As with the NBCA solutions, either 50 mg of MAG-oleate or 50 mg of MAG was added as well.

PLA/Ethyl Lactate Solutions.

A 425 mg portion of PLA solid was dissolved in 15 mL of ethyl lactate solvent. For the MAG-oleate solution, 50 mg of magnetic particles was added to 4.5 mL of PLA solution. For the MAG solution, 50 mg of particles was added to 2 mL of PLA solution.

Viscosity Measurements

Using a G.D.M. Couette Viscometer viscosity measurements were obtained for 2.5 mL samples of NBCA/Ethiodol® with MAG-oleate, CAIDMSO with MAG-oleate, CA/DMSO with MAG, PLA/ethyl lactate with MAG-oleate, PLA/ethyl lactate with MAG, and SBF (NBCA/Ethiodol® with MAG showed poor dispersion and thus was not measured). For three runs with each sample, viscosity in centipoise (cP) was measured at shear rates of 5 to 30 sec⁻¹ with increments of 1 sec⁻¹.

Dispersion Experiments.

In order to determine the dispersion of the magnetic materials in the polymer solutions, a qualitative settling test was conducted. A small amount of each sample, roughly 2 to 3 mL, was mixed thoroughly and allowed to settle. Observations for settling of magnetic particles were made periodically over a 180-minute period.

In Vitro Flow System Experiments.

To approximate performance within the actual clinical setting, each embolic formulation was evaluated in an in vitro dynamic flow system. This testing apparatus was adapted from Zambo, S. J. An In Vitro Testing Method for Embolic Materials used in Arteriovenous Malformation Therapy. Thesis. University of Florida, 1996, which is a modified version of a high flow rate circuit developed by Bartynski et al. [Radiology. 167:419-421 (1988)]. This system was used previously for quantitative pressure and flow measurement upon embolization of AVMs. In the parallel circuit design, one branch contains the vascular disease model and the other branch contains a resistor unit that models “normal” brain tissue beds. Pressure sensors are located at the inlets and outlets of the two branches and the flow sensor is located in the vascular disease branch. The function of the Starling resistor is to simulate the response by normal vascular beds to pressure and flow changes. The resistor consists of a rigid outer tube with a collapsible latex inner tube. The latex tube is pressurized hydrostatically with water. The flow sensor readings are of importance in determining the efficacy of the agents tested.

AVM and Aneurysm Model Construction.

An AVM model was constructed from polyurethane foam placed between two glass plates. Two portions of 0.25″ tubing serve as the feeding and draining side. Three wire insulation tubes serve as feeding vessels to the nidus. The feeding vessels and foam were encased by silicone. For the aneurysm model, the silicone was simply shaped as a saccular aneurysm roughly 8-10 mm in diameter.

Dynamic Testing of Embolic Materials.

The SBF was made using a procedure from Jungreis and Kerber [American Journal of Neuroradioloy. 12(2): 329-330 (March/April, 1991)]. First, 12.1 g of PVA was dissolved in one liter deionized water. In a separate container, 23.2 g of sodium borate was dissolved in deionized water. The two solutions were mixed and diluted to three liters. Boric acid was then added to lower the pH to 7.5. The system was prepped by first running the pump to filter any large particles and to clear any bubbles. The flow rate was set between 130 and 140 mL/min for the AVM model and around 100 to 110 mL/min for the aneurysm model. The Intramedic® 0.58 mm polyethylene tubing served as the microcatheter and was inserted into the flow system via the catheter introducer. A 0.3 tesla (3,000 gauss), 1″ by 1″ by 0.125″, Nd/Fe/B magnet (Edmund Optics, Barrington, N.J.) was placed on the AVM or aneurysm model 0.5 to 0.75 cm laterally from the direction of flow.

Each of the three polymer solutions was injected into the flow system with either an AVM or aneurysm model present for a total of five test runs (each polymer with either MAG-oleate or with MAG, excluding NBCA/Ethiodol®). Prior to injection, the catheter was rinsed with approximately 1 mL of 5% dextrose, DMSO, or ethyl lactate for NBCA, CA, and PLA, respectively. After thorough stirring, 1 to 2 mL of embolic solution was injected into the flow system. A Sony DCR-TR17 digital camera was used to capture the results.

Magnetic Measurements/Calculations.

The 0.3 tesla (3,000 gauss) magnet was used in calculations of the magnetic forces affecting the embolic agents. The magnetic field strength in gauss as a function of distance from the magnet was measured with a Gauss/Teslameter (Model 5080) from F. W. Bell®. The force due to the applied magnetic field was calculated as a function of distance from the magnet using the equation developed by Senyei et al., [Journal of Applied Physics. 49(6): 3578-3583 (June, 1978)].

### Viscosity Measurements

Qualitatively, upon injection into the flow system, all agents were able to be expunged but noticeable force was required, particularly with the CA with MAG-oleate. Each of the samples exhibits an amount of shear thinning, or decreasing viscosity with increasing shear rate. NBCA shows this trend only slightly compared to the other samples, although this result is not surprising considering that CA and PLA are polymer solutions. As increasing shear is applied to polymer solutions, the polymer chains begin to untangle and align in the direction of shearing. The resistance to flow, or viscosity, decreases as this untangling occurs. The viscosity of the NBCA solution is relatively constant at all shear rates because it is in monomer form.

The NBCA is a Newtonian fluid and follows the relationship τ=1 μ*du/dr, where τ is shear stress, μ is viscosity, and du/dr is the shear rate. From this equation, a plot of τ vs. du/dr gives a slope equal to μ. At the wall, du/dr is equal to 4Q/(π*R³) where Q is the flow rate and R is the diameter, such as the microcatheter diameter in this case. For a typical clinical injection rate of 1 cm³/30 sec and a microcatheter diameter of 0.2 mm, du/dr at the wall (where it is the greatest) is equal to 5300 s⁻¹ and for a Q of 1 cm³/45 sec is equal to 3500 s⁻¹.

Polymer solutions, such as PLA and CA, are typically non-Newtonian fluids and do not follow this relationship. For these fluids, the shear rate can be related to the shear stress by the power law equation:

$$τ = K \cdot {( \frac{ⅆ u}{ⅆ r} )}^{n}$$

The term K is the flow consistency index, and n is the flow behavior index (n<1 for shear-thinning fluids and n=1 for Newtonian ones). The shear rate at the wall can be described by the equation given below:

$$\frac{ⅆ u}{ⅆ r} = ( \frac{3 n + 1}{n} ) \cdot ( \frac{Q}{π {R}^{3}} )$$

From a plot (CA with MAG-oleate), n is approximated to be 0.45 by trial and error using the equation for shear stress above. For an injection rate of 1 cm³/30 sec, du/dr at the wall is equal to 6900 s⁻¹ and for a Q of 1 cm³/45 sec du/dr is equal to 4600 s⁻¹. For PLA with MAG-oleate, n is approximated to be 0.75, which corresponds to shear rates of 5700 s⁻¹ and 3800 s⁻¹, respectively for the previous flow rates.

These values for the shear rates of NBCA, CA, and PLA correspond to solution viscosities below the ideal upper limit of 20 cP (Zambo, supra). Thus these solutions are all effective for actual clinical injection. Many factors such as time, temperature, molecular weight, and concentration have an effect on the viscous behavior of non-Newtonian fluids. Accordingly, it will be understood by those skilled in the art that the optimum concentrations of the various components of the embolic agent of the invention will depend in each case on the nature thereof, their behavior in the presence of each other and the intended mode of delivery.

### Dispersion Testing

The qualitative results for the dispersion tests can be seen in Table 1. As seen from the data, the MAG-oleate samples showed a greater degree of dispersion exhibited by the longer time for settling. These samples did not completely settle until hours later. The considerable smaller size of the MAG-oleate compared to MAG is considered the leading reason for this observation.

Table 1 Settling of magnetite in the various polymer solutions.

KEY: 1 completely black solution; 5-clear solution with all particles on bottom

TABLE 1
Settling of magnetite in the various polymer solutions.

| | NBCA | | CA - | | PLA | |
|---|---|---|---|---|---|---|
| | w/MAG- | NBCA | w/MAG- | CA | w/MAG- | PLA |
| | oleate | w/MAG | oleate | w/MAG | oleate | w/MAG |
| 5 min | 1 | No | 1 | 1 | 1 | 1 |
| | | dispersion | | | | |
| 10 min | 1 | — | 1 | 1 | 1 | 1 |
| 20 min | Polymer- | — | 1 | 1 | 1 | 1 |
| | ization | | | | | |
| | beginning | | | | | |
| 30 min | Polymer- | — | 1 | 1 | 1 | 1 |
| | ized | | | | | |
| 60 min | — | — | 1-2 | 1-2 | 1 | 2-3 |
| | | | cloudy layer | cloudy layer | | cloudy layer |
| | | | at top | at top | | at top |
| 90 min | — | — | 1-2 | 2-3 | 1-2 | 3 |
| | | | | magnetite | | magnetite |
| | | | | layer on | | layer on |
| | | | | surface | | surface |
| 120 | — | — | 1-2 | 2-3 | 2 | 4 |
| min | | | | | | |
| 150 | — | — | 2 | 3. | 2 | 4-5 |
| min | | | slightly | | | nearly all |
| | | | cloudier | | | settled |
| 180 | — | — | 2 | 3 | 2 | 4-5 |
| min | | | | slower | | |
| | | | | settling | | |

KEY:
1 completely black solution; 5 -clear solution with all particles on bottom

Interestingly, the NBCA with MAG was unable to be mixed. The particles merely clumped together and no mixing was evident. As a result this agent was not tested for viscosity or in the flow system.

Although a noticeable difference is noted in the settling times between the samples, none of the samples precipitated at such a rate that would cause concern in actual clinical use. Even 30 minutes after mixing, all solutions were still fairly well dispersed.

### In Vitro Dynamic Testing of Magnetic Embolic Agents

In order to visualize the performance of the embolic agents, glass plates were used to allow digital taping of injections into the AVM and aneurysm models. Although many approximations are made using these models, their function is satisfactory for the purpose of evaluating efficacy.

NBCA/Ethiodol® Solutions

Due to poor dispersion with the non-oleate-coated magnetite, only the NBCA formulation with oleate-coated magnetite was tested. Initially, the NBCA was mixed with Ethiodol® and the MAG-oleate. Upon stirring the mixture polymerized within 1 to 2 minutes. Hence, the addition of glacial acetic acid (GAA) was needed to slow the polymerization by reducing the interaction with basic ion species found on the MAG-Oleate. Mixtures were made at 1%, 3%, and 6% GAA. The results showed that 3% GAA was able to prevent any pre-injection polymerization over approximately a 20-minute time period. The 1% mixture polymerized within 2 to 3 minutes. The 3% formulation was thus used for injection into the flow system.

Digital photo results for injection of 1 Ml of NBCA into an AVM model are seen in FIG. 1. The flow rate for the run was 115 mL/min. Injection of NBCA was relatively easy, requiring only slight effort to depress the syringe. This observation agrees with the relatively low measured viscosity for NBCA. The material appeared to exit the microcatheter in globular form as opposed to a stream of material. The embolic agent responded very favorably to the magnetic field and migrated swiftly to the magnet.

The glue appeared to polymerize fairly rapidly and no material was detected traveling out of the model and passing downstream. The solid mass showed good coherence, exhibited by no flakiness from the GAA, which had been previously noted by Zambo, supra.

CA/DMSO Solutions

Both MAG-oleate and MAG dispersed well in the CA and PLA solutions. Digital photo results are shown in FIGS. 2-5. The flow rates for the models were 140, 100, and 130 mL/min, respectively. Approximately 1.5-2 mL of solution was injected and a greater force was needed to depress the syringe compared to NBCA. The fluid exited the catheter in a stream and redirected nicely to the magnet. The polymers formed a soft, solid mass concentrated on the magnet. However, some unintentional overfilling by the user occurred in the MAG aneurysm (CA). For the AVM models, material began to flow well to the magnet.

Overall the PLA showed promise as a new embolic agent. Taking advantage of the properties of PLA polymer gives rise to a different approach for the treatment of vascular lesions by promoting a healing by the body. One example would be the induction of fibrosis by release of a fibroblast growth factor over time [(Hong et al., Neurosurgery. 49(4): 954-961 (October, 2001)].

Magnetic Force Calculations

The measurements for the magnetic field strength with respect to distance from the magnet are presented in Table 2.

In order to approximate the magnetic force acting on the embolic agents, the other variables in the force equation must be calculated or estimated. The magnetic field gradient, dH/dx in units of A/m², was approximated (values given in Table 2). The magnetization values were calculated from the density of magnetite, 5.17 g/cm₃, and from the magnetization curve due to an applied field for the MAG-oleate magnetite. The values of M, in units of A/m, are also shown in Table 2. The radius value is estimated as the radius of the solidified mass after infection, or approximately 1.5 cm. The volume fraction is approximated at 0.01 from visual inspection of the settled magnetite layer, the mass of MAG-oleate added, and the density of magnetite. Using these values, the force is calculated (Table 2).

The magnetic field and force effects due to the field fall off rapidly with distance. Of course in this study, only a very weak, 0.3 tesla magnet was used and the values would be much different for a stronger magnetic source. An external magnetic source, such as an MRI unit with field strengths in several tesla, could be utilized in the practice of the invention to provide the magnetic guidance of these embolic agents.

TABLE 2
Values for measured magnetic field strengths and calculated forces.

| | | | | 71 |
|---|---|---|---|---|
| Distance | Field Strength | dH/dx | M | F_{M} |
| (cm) | (gauss) | (A/m²) | (A/m) | (N) |
| 0 | 2400 | 3.184 × 10⁷ | 3.1 × 10⁵ | 1.75 |
| 0.5 | 400 | 2.125 × 10⁷ | 1.55 × 10⁵ | .585 |
| 1 | 190 | 1.99 × 10⁶ | 1.03 × 10⁵ | .0364 |
| 1.5 | 105 | 1.083 × 10⁶ | 8.79 × 10⁴ | .0169 |
| 2 | 55 | 5.49 × 10⁵ | 7.76 × 10⁴ | .00756 |
| 2.5 | 35 | 3.98 × 10⁵ | 6.72 × 10⁴ | .00475 |
| 3 | 20 | 2.55 × 10⁵ | 6.2 × 10⁷ | .0021 |
| 3.5 | 15 | 1.19 × 10⁵ | 5.17 × 10⁴ | .00109 |
| 4 | 10 | 8.76 × 10⁴ | 4.14 × 10⁴ | .000644 |
| 4.5 | 6 | 6.37 × 10⁴ | 3.1 × 10⁴ | .000351 |
| 7 | 1 | — | — | — |
| 8.5 | 0 | — | — | — |

The development of the above three different magnetic embolic agents was accomplished using oleate-coated and non-oleate-coated magnetite as the magnetic component for each. The viscous and dispersive properties of these agents are close to ideal and are capable of injection with little problem and no settling of magnetic material prior to injection. Particle size and aggregation appear to be the biggest factors for the dispersive differences between the coated and non-coated magnetite.

Digital photo results show that all of the agents were successfully delivered in either an AVM or aneurysm model under the influence of a magnetic field. NBCA did seem to be superior to the other two solutions in terms of coherence. The use of a magnetic NBCA is also particularly interesting because NBCA is already in widespread use. By the addition of oleate-coated magnetite, according to the invention, and, optionally GAA, the material, under direction of a magnetic field, can be used to occlude AVMs and aneurysms.

CA and PLA are also attractive in the practice of the invention considering the thrombogenic, relatively non-toxic properties of CA, particularly for promoting thrombosis and fibrosis of aneurysms. PLA, a biodegradable material, can be infused with drugs or other factors such as fibrin to promote occlusion of the aneurysm by fibrosis. Indeed, any physiologically compatible bioactive agent, such as a drug, for example, may be incorporated in the embolic composition. Any suitable such agent, such as a drug may be incorporated in the implants of the invention depending in each case, of course, on the intended use and application of the prosthesis. Exemplary of such drugs are, an anti-inflammatory agent such as dexamethasone, methotrexate, an immunosuppressive agent such as siroilmus, an interleukaus such as IL-lO, a cell wall lipid such as MPL, a cytotoxic agent such as taxol, mitoxantrone, 5-FU, ara-C or mixtures thereof.

The term “drug” as used herein is intended to include drugs, pharmaceutical compounds, therapeutic agents, anti-microbial or anti-bacterial compounds, proteins, peptides, plasmids and gene therapy agents/compounds and bioactive compounds/substances.

The viscosity measurements of the above samples were determined and NBCA was found to be the only material with a viscosity below the desired threshold of 20 cP at all shear rates. The other materials showed shear thinning, non-Newtonian behavior. However, the shear rates present at typical clinical injection flow rates, correlate to viscosities below 20 cP for CA and PLA with and without MAG-oleate. However, the viscosity and, as a result, the force necessary for injection will be greatest at the beginning of injection for these fluids, and the incremental increase in force will be less at higher injection pressures, corresponding to higher shear rates of the fluid. A plug of higher viscosity fluid is also present near the center of the microcatheter lumen where the shear rate linearly approaches zero. Accordingly, if the forces for injection are too great, a larger diameter microcatheter may be used or the composition of the solutions can be adjusted to achieve satisfactory results.

The biodegradable compositions can be used for drug delivery to hard to reach places such as brain tumors, infections, epileptogenic foci, centers of motor disturbance (such as areas treated with deep-brain stimulation), and other locations.

From the foregoing description, various modifications and changes in the composition and method will occur to those skilled in the art. All such modifications coming within the scope of the appended claims are intended to be included therein. The entire disclosures and contents of each and all references cited and discussed herein are expressly incorporated herein by reference. All percentages expressed herein are by weight unless otherwise indicated.

1. A flowable, biodegradable endovascular embolic composition effective for embolizing a vascular defect consisting essentially of:
(a) a biocompatible, biodegradable polymer or polymeric material forming composition;
(b) a biocompatible embolic solvent for said polymer or polymer forming composition;
(c) biocompatible magnetic particles responsive to a magnetic field;
wherein:
said polymer or polymeric forming material and solvent are present in said composition in amounts and relative proportions such that (1) said composition is deliverable to a vascular defect site and (2) upon delivery to said site, solidifies into an embolic mass; and
said magnetic particles are present in said composition in an amount sufficient to enable said composition being deliverable to said vascular site by a magnetic field.

2. The composition of claim 1 wherein said polymer or polymeric material forming composition is present in an amount of 2% to 50%, by weight for polymer.

3. The composition of claim 1 wherein said solvent is present in an amount of 5% to 60%, by weight.

4. The composition of claim 1 wherein said magnetic particles are present in an amount of 2% to 50%, by weight.

5. The composition of claim 1 wherein said polymer or polymeric material forming composition is polylactic acid, glycolic acid (PLGA), polycaprolactone or polyglutamate esters.

6. The composition of claim 1 wherein said solvent is DMSO, ethanol, ethyl lactate, acetone, N-methylpyrrolidone, ethylene glycol ethers (e.g., ethylene glycol dimethyl ether, or di-ethylene glycol dimethyl ether.

7. The composition of claim 1 wherein said magnetic particles are magnetite (Fe₃O₄), maghemite, or iron sulfur minerals.

8. The composition of claim 1 wherein said magnetic particles are coated with a biocompatible surfactant.

9. The composition of claim 1 wherein said surfactant is a fatty acid or salt thereof.

10. The composition of claim 8 wherein said fatty acid is an 18 carbon atom acid.

11. The composition of claim 9 wherein said fatty acid is oleic acid.

12. The composition of claim 10 wherein said magnetic particles are coated with sodium oleate.

13. The composition of claim 1 also containing a radiopaque agent.

14. The composition of claim 1 wherein said radiopaque agent is barium sulfate, potassium iodide, an organic iodine containing molecule such as thyroxine.

15. The composition also including a bioactive agent for sustained release from said embolic mass.

16. The composition of claim 12 wherein said bioactive agent is a drug or medicant.

17. A method of treating a vascular defect comprising introducing the composition of claim 1 into the vascular defect under the guidance of a magnetic field and positioning the composition in said vascular defect with said magnetic field under conditions wherein and until said composition solidifies into an embolic mass.

18. An article of manufacture comprising the composition of claim 1.

19. The article of manufacture of claim 15 comprising packaging material and an embolic composition contained within said packaging material, wherein said embolic composition is effective for embolizing a vascular defect utilizing an applied magnetic field, and wherein said packaging material comprises a label which indicates that said embolic composition can be used for treating vascular defects and is deliverable to said vascular defect by an applied magnetic field, and wherein said embolic composition is that of claim 1.
